(19) United States
(12) Patent Application Publication
(10) Pub. No.: US 2008/0108071 A1
(43) Pub. Date: May 8, 2008

(54) Methods and Systems to Determine Fetal Sex and Detect Fetal Abnormalities

(76) Inventor: Katherine Thompson, Houston, TX (US)

Correspondence Address: BAKER BOTTS, LLP, 910 LOUISIANA, HOUSTON, TX, 77002-4995, US

(21) Appl. No.: 11/869,064

(22) Filed: Oct. 9, 2007

Related U.S. Application Data
(60) Provisional application No. 60/828,897, filed on Oct. 10, 2006.

(51) Int. Cl. C12Q 1/68; A61B 8/13

(52) U.S. Cl. 435/6; 600/443

(57) ABSTRACT

Non-invasive methods for determining the sex of a human fetus and predicting other genetic abnormalities are disclosed. The methods include screening a maternal sample for biomarkers known to be associated with risk of genetic abnormalities; removing all or substantially all nucleated and anucleated cell populations from the maternal sample to obtain a remaining material; detecting in the remaining material, the presence of nucleic acid; and determining the sex of the fetus from the nucleic acid wherein the presence of a certain marker is indicative of a male fetus; performing an ultrasound scan which yields quantitative measurements of the fetus; and interpreting the results of the genetic abnormality screening in conjunction with the ultrasound measurements.

### CROSS REFERENCE TO RELATED APPLICATIONS

This application claims the benefit of U.S. Provisional Application Ser. No. 60/828,897 filed Oct. 10, 2006, which is incorporated by reference herein.

### BACKGROUND

The present invention relates generally to a method of screening a maternal sample for the purpose of determining the gender of the pregnant woman's fetus and for predicting fetal abnormalities and pregnancy outcomes.

Conventional prenatal screening methods for detecting fetal abnormalities and for sex determination traditionally use fetal samples derived by invasive techniques such as amniocentesis and chorionic villus sampling. These techniques require careful handling and present a degree of risk to the mother and to the pregnancy.

The presence of DNA originating from different individuals in bodily fluids is a well-known biological phenomenon in many clinical and biological scenarios. During pregnancy, detection of fetal DNA in maternal plasma and serum has been previously demonstrated. This technology has demonstrated that fetal DNA isolated from maternal plasma and serum can be used for non-invasive prenatal diagnosis. The clinical application of this phenomenon has been helped by the relatively high absolute and relative concentrations of such circulating fetal DNA in maternal plasma and serum. Using this approach, noninvasive prenatal detection of a number of conditions has been achieved, including fetal rhesus D status, myotonic dystrophy, achondroplasia, and certain chromosomal translocations. All of these approaches have utilized the detection of DNA sequences inherited from the father and which are genetically distinguishable from those of the mother. Specifically, the detection of DNA that the fetus has inherited from the mother in maternal plasma or serum has been thought to be impossible. Similar limitations have also been described for the detection of fetal nucleated cells isolated from the cellular fraction of maternal blood.

Techniques which use maternal blood or serum samples have also been devised for predicting abnormalities in the fetus and possible complications in pregnancy. Three markers commonly used include alpha-foetoprotein (AFP—of fetal origin), human chorionic gonadotrophin (hCG), and estriol, for screening for Down's Syndrome and neural tube defects. Maternal serum is also currently used for biochemical screening for chromosomal aneuploidies and neural tube defects. The passage of nucleated cells between the mother and fetus is now a well-recognized phenomenon. The use of fetal cells in maternal blood for non-invasive prenatal diagnosis avoids the risks associated with conventional invasive techniques. Prenatal genetic determination using fetal DNA obtained from fetal cells in the maternal blood has been reported. Considerable advances have been made in the enrichment and isolation of fetal cells for analysis. However, these techniques are time-consuming or require expensive equipment.

Noninvasive prenatal molecular diagnosis has been performed on the fetal cells of cell-free fetal DNA that are released into the maternal circulation during pregnancy. The concentration of the cell-free fetal DNA is about 3.4% (0.39%-11.9%) and 6.2% (2.33%-11.4%) of the total plasma DNA during early and late pregnancy respectively, which can be detected by sensitive PCR assay. Prenatal diagnosis on fetal DNA has been successfully performed for fetal gender determination and a variety of single gene disorders.

Several biochemical markers are under investigation as screening markers for fetal disease and adverse pregnancy outcome, for example, Down's syndrome, and other chromosomal diseases in early pregnancy. One that has come into routine use is pregnancy-associated plasma protein-A (PAPP-A), which has also been shown to be of potential clinical importance as a marker of growth retardation and preterm birth. ADAM12 has also been the target of screenings for abnormal cell function.

Commercially, the UltraScreen® test has been marketed as a predictor of fetal abnormalities. The test relies on a combination of screening for certain biomarkers, an ultrasound measurement, and maternal information to diagnose the fetus. The UltraScreen test does not determine fetal gender, which is relevant to the determination of whether a fetus is susceptible to certain X-linked disorders.

### SUMMARY

The methods of the present disclosure generally comprise a test or set of tests performed on a sample from a pregnant woman and further generally comprise a quantitative ultrasound of the fetus. The general object of the disclosed invention is to determine multiple factors about the fetus and the pregnancy, including the sex of the fetus, and possible genetic abnormalities and pregnancy outcomes. The test which is used to determine sex is performed on cell-free DNA in a sample of body fluid from the pregnant woman. Tests for other biochemical markers associated with fetal abnormalities and pregnancy outcomes may be performed on the cell-free DNA or another fraction of the sample. The method also comprises an ultrasound of the fetus which provides further quantitative data to interpret in conjunction with the results of the other tests.

In certain embodiments, methods for prenatal screening are provided that comprise screening cell-free fetal DNA found in a maternal sample to determine the sex of the fetus; screening a maternal sample for other biochemical markers that have been found to be related to fetal abnormalities or pregnancy outcomes; and using measurements from an ultrasound scan of a fetus to confirm or aid in the interpretation of the screening tests.

In one aspect of the disclosed methods, gender determination may be accomplished through a screen for the Y chromosome in the fetal DNA. In the preferred embodiment, the Y chromosome is detected by screening for the SRY marker, unique to the Y chromosome. The presence of SRY is interpreted as a determination that the fetus is male.

In addition to the gender determination test, another aspect of the disclosed methods is a screen for biochemical markers associated with fetal abnormalities, particularly biochemical markers for Down's syndrome. While many biochemical proteins can be used in prenatal testing, the goal in selecting biochemical markers is to obtain the best balance between detection efficiency and false positive rate. This is accomplished by picking markers with the greatest difference between levels found in affected and unaffected pregnancies. For Down's Syndrome pregnancies, these two proteins are widely accepted to be PAPP-A (pregnancy associated plasma protein) and the free-lying beta subunit of hCG (freeBeta™). Human chorionic gonadotropin (hCG) is comprised of an alpha and beta subunit, with the beta subunit produced in significant excess in Down's Syndrome pregnancies. PAPP-A (Pregnancy Associated Plasma Protein) is another marker with a significant difference in levels seen between a normal pregnancy and a Down's syndrome affected pregnancy. Screening for these two biochemical analytes alone (without any accompanying ultrasound measurements) allows for a 65% detection of Down's syndrome in the first trimester.

Another aspect of the disclosed methods is taking measurements from an ultrasound scan, for example to measure nuchal translucency (“NT”). Nuchal translucency is defined as the space from the back of the fetal neck to the skin overlying the neck and it refers to an observation that abnormal fetuses tend to show an accumulation of fluid in this region and have an increased risk of having a variety of chromosome abnormalities present, including the common finding of Down's Syndrome, or Trisomy 21. After this finding was first reported, an extended study was initiated by Kypros Nicolaides in London, England which demonstrated that through using an increased NT measurement, 78% of Down's syndrome pregnancies could be detected with a 5% false positive rate.

When the NT measurement may be combined with screening for these two biochemical markers, between 85-91% of Down syndrome pregnancies can be detected between 11-14 weeks of pregnancy as well as 40% detection of cardiac abnormalities and greater than 97% detection of other chromosome abnormalities. This compares very favorable to the 65-70% detection rate seen in the current method of serum screening using the triple screen of hCG, AFP, and estriol.

Accordingly, the present invention combines the noninvasive cell-free DNA gender determination test and other screening tests from a maternal sample. The results of those diagnostic tests are interpreted in conjunction with or supplemented by ultrasound measurements of the fetus' physical structure.

Further objects of the invention are to produce an apparatus and a computer program product for automating the ultrasound portion of the screening procedure.

The features and advantages of the present disclosure will be readily apparent to those skilled in the art upon a reading of the description of exemplary embodiments, which follows.

### DESCRIPTION

The present disclosure relates to methods for determining the sex of a fetus, predicting abnormalities in the fetus, and predicting possible pregnancy complications. More particularly, the present disclosure relates to the combination of methods of screening maternal plasma or serum for cell-free fetal DNA to determine the sex of the fetus and screening the maternal sample for other biochemical markers to gather more information about the fetus, including the possibility of genetic abnormalities. The results of these screenings are interpreted in combination with a quantitative ultrasound test, which may supplement, confirm, or contradict the results of the screening performed on the maternal sample.

Preparing Maternal Samples for Screening

Generally, the maternal sample is maternal serum or a plasma sample from a pregnant female. As used herein, the term “maternal sample” is intended to encompass any fluid or cellular sample or mixture thereof obtained from a pregnant female. Specifically, the term includes tissue biopsy, serum, plasma, or amniotic fluid samples. In one embodiment, the maternal serum or plasma sample is derived from the maternal blood. As little as 10 μL of serum or plasma can be used. However it may be preferable to employ larger samples in order to increase accuracy. The volume of the sample required may be dependent upon the condition or characteristic being detected. In any case, the volume of maternal blood which needs to be taken is small.

The preparation of serum or plasma from the maternal blood sample is carried out by standard techniques. The serum or plasma is normally then subjected to a nucleic acid extraction process. A suitable method includes the method described herein in the example, and variations of that method. Possible alternatives include the controlled heating method described by Frickhofen and Young (1991). Another suitable serum and plasma extraction method is proteinase K treatment followed by phenol/chloroform extraction. Serum and plasma nucleic acid extraction methods allowing the purification of DNA or RNA from larger volumes of maternal sample increase the amount of fetal nucleic acid material for analysis and thus improve the accuracy. A sequence-based enrichment method could also be used on the maternal serum or plasma to specifically enrich for fetal nucleic acid sequences.

In other embodiments, the DNA species are differentiated by observing epigenetic differences in the DNA species such as differences in DNA methylation. As used herein, the term “epigenetic difference” is intended to encompass any molecular or structural difference other than the primary nucleotide sequence. For instance, in situations where one DNA species comes from a male, and one DNA species comes from a female, the epigenetic marker may be the inactivated X chromosome of the female individual. In such embodiments, methylated DNA sequences on the inactivated X chromosome may be used to detect DNA originating from the female individual. In some embodiments, the epigenetic differences may be analyzed inside cells. Further, in some embodiments, the epigenetic differences may be analyzed using in-situ methylation-specific polymerase chain reaction. Additionally, the epigenetic differences may be used to sort or isolate cells from the respective individuals or to purify DNA from the respective individuals. The methods according to the present invention may be performed with or without measuring the concentrations of DNA species, however, in preferred embodiments, the concentrations of DNA species with the respective epigenetic differences are measured. Such measuring of concentrations involves measuring the respective DNA methylation differences in embodiments wherein DNA methylation differences is the epigenetic marker. In some embodiments, sodium bisulfite is added to the biological sample or to the DNA species directly to detect the DNA methylation differences. However, in other embodiments a methylation-specific polymerase chain reaction, as is well known to those skilled in the art, may be used to detect the DNA methylation differences. In yet other embodiments, DNA sequencing or primer extension may be used to detect the methylation differences.

An amplification of fetal DNA sequences in the sample is normally carried out. Standard nucleic acid amplification systems can be used, including PCR, the ligase chain reaction, nucleic acid sequence based amplification (NASBA), branched DNA methods, and so on. Preferred amplification methods involve PCR.

Screening the Maternal Sample to Determine the Sex of the Fetus

The methods of the present disclosure are used to determine the sex of the fetus, which may be carried out by detecting the presence of a marker which is specific to the Y chromosome. In one embodiment, the preferred marker is the SRY gene. It has previously been demonstrated that using only 10 μL of plasma or serum a detection rate of 80% for plasma and 70% for serum can be achieved. The use of less than 1 mL of maternal plasma or serum has been shown to give a 100% accurate detection rate.

It is anticipated that it will be possible to incorporate the nucleic acid-based diagnosis methods described herein into existing prenatal screening programs. Sex determination has successfully been performed on pregnancies from 10 to 14 weeks of gestation.

Screening the Maternal Sample to Detect Fetal Abnormalities

In some embodiments, the methods of the present disclosure can be applied to the detection of any paternally-inherited sequences which are not possessed by the mother and which may be genes which confer a disease phenotype in the fetus. Examples include:

(a) Fetal rhesus D status determination in rhesus negative mothers (Lo et al 1993). This is possible because rhesus D positive individuals possess the rhesus D gene which is absent in rhesus D negative individuals. Therefore, the detection of rhesus D gene sequences in the plasma and serum of a rhesus D negative mother is indicative of the presence of a rhesus D positive fetus. This approach may also be applied to the detection of fetal rhesus D mRNA in maternal plasma and serum.

(b) Haemoglobinopathies (Camaschella et al 1990). Over 450 different mutations in the beta-globin gene have been known to cause beta-thalassaemia. Provided that the father and mother carry different mutations, the paternal mutation can be used as an amplification target on maternal plasma and serum, so as to assess the risk that the fetus may be affected.

(c) Paternally-inherited DNA polymorphisms or mutations. Paternally-inherited DNA polymorphisms or mutations present on either a Y or a non-Y chromosome can be detected in maternal plasma and serum to assess the risk of the fetus being affected by a particular disease by linkage analysis. Furthermore, this type of analysis can also be used to ascertain the presence of fetal nucleic acid in a particular maternal plasma or serum sample, prior to diagnostic analysis such as sex determination. This application will require the prior genotyping of the father and mother using a panel of polymorphic markers and then an allele for detection will be chosen which is present in the father, but is absent in the mother.

The plasma or serum-based non-invasive prenatal diagnosis method according to the invention can be applied to screening for Down's Syndrome, and other chromosomal aneuploidies. The term “prenatal diagnosis” as used herein covers determination of any maternal or fetal condition or characteristic which is related to either the fetal DNA itself or to the quantity or quality of the fetal DNA in the maternal serum or plasma. Included are sex determination, and detection of fetal abnormalities which may be for example chromosomal aneuploidies or simple mutations. Also included is detection and monitoring of pregnancy-associated conditions such as pre-eclampsia which result in higher or lower than normal amounts of fetal DNA being present in the maternal serum or plasma. The nucleic acid detected in the method according to the invention may be of a type other than DNA, for example mRNA. Two possible ways in which prenatal diagnosis might be done are as follows:

(i) It has been found that in pregnancy involving fetuses with chromosomal aneuploidies, for example Down's Syndrome, the level of fetal cells circulating in maternal blood is higher than in pregnancies involving normal fetuses. Following the discovery that fetal DNA is present in maternal plasma and serum, it has also been demonstrated that the level of fetal DNA in maternal plasma and serum is higher in pregnancies where the fetus has a chromosomal aneuploidy than in normal pregnancies. Quantitative detection of fetal nucleic acid in the maternal plasma or serum, for example a quantitative PCR assay, can be used to screen pregnant women for chromosomal aneuploidies.

(ii) A second method involves the quantitation of fetal DNA markers on different chromosomes. For example, for a fetus affected by Down's Syndrome the absolute quantity of fetal chromosomal 21-derived DNA will always be greater than that from the other chromosomes. The recent development of very accurate quantitative PCR techniques, such as real time quantitative PCR (Heid et al 1996) facilitates this type of analysis.

Another application of the accurate quantitation of fetal nucleic acid levels in the maternal serum or plasma is in the molecular monitoring of certain placental pathologies, such as pre-eclampsia. The concentration of fetal DNA in maternal serum and plasma is elevated in pre-eclampsia. This is probably due to the placental damage which occurs.

In one embodiment, the present disclosure features methods of detecting abnormalities in a fetus by detecting fetal DNA in a biological sample obtained from a pregnant woman. The methods according to this embodiment provide for detecting fetal DNA in a maternal sample by differentiating the fetal DNA from the maternal DNA based upon epigenetic markers such as differences in DNA methylation. Employing such methods, fetal DNA that is predictive of a genetic anomaly or genetically based disease may be identified thereby providing methods for prenatal diagnosis. These methods are applicable to any and all pregnancy-associated conditions for which methylation changes associated with a disease state is identified. Exemplary diseases that may be diagnosed include, for example, preeclampsia, a chromosomal aneuploidy, including but not limited to trisomy 21, Prader-Willi Syndrome, and Angelman Syndrome.

As with the broader differentiating methods of the first aspect of the invention, the biological sample obtained from the mother is preferably plasma or serum. The differentiation between maternal and fetal DNA may be performed with or without quantifying the concentration of fetal DNA in maternal plasma or serum. In embodiments wherein the fetal DNA is quantified, the measured concentration may be used to predict, monitor or diagnose or prognosticate a pregnancy-associated disorder. In some embodiments, the particular fetus-derived epigenetic mark is associated with a fetal disorder, and in some embodiments an epigenetic characteristic in fetal cells in the placenta is used as a fetus-specific marker in maternal plasma or serum.

According to a certain set of embodiments, the aim of the fetal abnormalities screening test is to identify women who are at a sufficiently high risk of Down's syndrome to justify a further test which is diagnostic of Down's syndrome. Such further diagnostic tests, for example chorionic villus sampling or amniocentesis, involve sampling procedures that carry a certain risk to the mother and/or fetus, the induction of miscarriage and fetal limb defects being among the recognized hazards. There is, therefore, a need for screening tests that maximize the chance of identifying those pregnancies at highest risk of Down's syndrome, so as to justify further diagnostic tests with their attendant risks.

The effectiveness of a screening test depends on its ability to discriminate between pregnancies with Down's syndrome and unaffected pregnancies. The discriminatory power of a test is usually specified in terms of the detection rate achieved for a given false-positive rate, or in terms of the false-positive rate required to achieve a given detection rate. The detection rate is the proportion of Down's syndrome pregnancies with a positive result. The false-positive rate is the proportion of unaffected pregnancies with a positive result.

Different screening markers generally impart more discriminatory power to a screening test at one stage of the pregnancy than at other stages. In the preferred embodiment, the screening test relies on certain combinations of biochemical and ultrasound markers that have been identified as being effective when used together at a specific, single stage of pregnancy. For example, the “combined test” carried out in the first trimester using nuchal translucency and free P-hCG and PAPP-A as screening markers can achieve an 80% detection rate with a 5% false-positive rate.

Measurements carried out on biochemical samples may include assaying one or more of the following biochemical markers of Down's syndrome in maternal serum or plasma, among others: alpha feto-protein (AFP), unconjugated oestriol (uE₃), human chorionic gonadotrophin (hCG), free alpha sub-unit of hCG (free a-hCG), free beta sub-unit of hCG (free P-hCG), inhibin, preferably dimeric inhibin-A (inhibin A), and pregnancy-associated plasma protein A (PAPP-A).

Ultrasound Measurements to Supplement the Results of Screening

The methods of the present disclosure further comprise an ultrasound scan which produces quantitative physical measurements of the fetus. In one embodiment, these measurements are interpreted in conjunction with the gender determination screening and genetic abnormality screening to assess risk of fetal abnormalities and pregnancy outcomes. In general, an NT measurement is taken through ultrasound and interpreted in conjunction with the results of a maternal sample screened for free P-hCG and PAPP-A.

Other embodiments of the disclosed invention may take into account other measurements carried out on ultrasound images, including one or more of the following ultrasound markers of Down's syndrome, among others: nuchal translucency (NT) thickness, nuchal fold thickness, and fetal femur length. Other physical measurements that may be considered in combination with the genetic abnormality screening include humerus, cephalic index, ventricular size, and ultrasound detected defects in the heart, gut, and organs, among other physical markers. Use of these and other screening markers at a single stage of pregnancy is known, so the specific techniques by which measurements are obtained need not be described in detail here.

Automation

The processing of the measurements of the screening marker levels may be implemented by an appropriately programmed computer. In another implementation, these computer-implemented steps may be provided as an article of manufacture, that is, in accordance with an aspect of the present invention, a computer program storage medium readable by a computing system and encoding a computer program of instructions for executing a computer process for determining a pregnant woman's risk of having a fetus with a genetic abnormality, the computer process comprising the steps of: inputting the sex of the fetus, a measurement of at least one screening marker level associated with a genetic abnormality or pregnancy outcome, and ultrasound measurements. The computer-implemented steps may also be provided as a machine, including modules for performing the processing.

To facilitate a better understanding of the present invention, the following examples of specific embodiments are given. In no way should the following examples be read to limit or define the entire scope of the invention.

### EXAMPLES

The fetal gender was determined through a real-time TaqMan PCR assay at a gestation age of 10-14 weeks. The experimental procedure began with a maternal blood sample that was centrifuged at 3000 rpm for 10 minutes. The plasma was removed and transferred to a 15 ml polypropylene tube, taking great caution not to disturb the buffy coat layer. The plasma was then centrifuged again at 3000 rpm for 10 minutes and the supernatant was collected for further analysis. At that point, the plasma may have been stored at −20° C.

In the next step, the cell-free fetal DNA was extracted from the plasma using the QIAGEN Micro kit. The samples were processed in the following order: the plasma sample, two positive sample controls, and a reagent blank. 70 μL of protease were added to and mixed with 700 μL of plasma sample. Steps 3-10 of the Blood and Body Fluid Spin Protocol of the QIAamp DNA Blood Mini Kit Handbook were followed, including Step 9a. Finally, the DNA was eluted in 15 μL AE buffer.

The samples were then prepared for PCR amplification. The 5 μM primer used for the PCR contained 900 μL H₂O, 50 μL SRY-109F forward primer at stock concentration 100 μM, and 50 μL SRY-245R reverse primer at stock concentration μM. The 2.5 μM probe contained 195 μL 1 H2O and 5 μL of SRY-142T probe at stock concentration 100 μM. For each 50 μL reaction, PCR master mix was prepared by mixing 25 μL Taqman Master Mix, 10 μL Primer (5 μM) and 5 μL Probe (2.5 μM). 40 μL of PCR master mixture was aliquoted into a 96-well plate and 10 μL of DNA was added to the wells to create 50 μL samples. The samples were run in an ABI 7300 Real Time PCR System according to the following profile: 50° C. for 2 minutes, 95° C. for 10 minutes, 50 cycles of 95° C. for 10 seconds and 60° C. for 1 minute, and 60° C. for 1 hour.

When the PCR was complete, the results were interpreted. The presence of an amplification curve in the experimental DNA sample was interpreted as a positive result that the fetus was male. The results were later compared with the results of the ultrasound scan to confirm the sex of the fetus.

The experimental procedure was performed on 203 pregnant females in the first trimester of pregnancy. For 202 females, the screening results for both experimental samples were in agreement about the sex of the fetus, and that sex was later confirmed in the second trimester through an ultrasound scan. For one of the pregnant females, the screening result for one of the experimental samples indicated that the fetus was a boy and the other experimental sample indicated that the fetus was a girl. When the procedure was repeated on the sample, the result indicated that the fetus was a boy. However, the later ultrasound indicated that the fetus was female.

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

What is claimed is:

1. A non-invasive method for determining the sex of a human fetus and predicting other genetic abnormalities, comprising: screening a maternal sample for biomarkers known to be associated with risk of genetic abnormalities; removing all or substantially all nucleated and anucleated cell populations from the maternal sample to obtain a remaining material; detecting in the remaining material, the presence of nucleic acid; and determining the sex of the fetus from the nucleic acid wherein the presence of a certain marker is indicative of a male fetus; performing an ultrasound scan which yields quantitative measurements of the fetus; and interpreting the results of the genetic abnormality screening in conjunction with the ultrasound measurements.

2. The method according to claim 1 wherein the remaining material comprises plasma.

3. The method according to claim 1 wherein the remaining material comprises serum.
